(19) United States
(12) Patent Application Publication
(10) Pub. No.: US 2006/0141458 A1
(43) Pub. Date: Jun. 29, 2006

(54) Method of identifying genes involved in cell growth regulation

(76) Inventor: Jan-Elo Jorgensen, Skanderborg (DK)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/522,121

(22) PCT Filed: Jul. 18, 2003
(86) PCT No.: PCT/DK03/00504

§ 371 Date: Sep. 29, 2005

Related U.S. Application Data
(60) Provisional application No. 60/397,761, filed on Jul. 22, 2002.

(51) Int. Cl. C12Q 1/68

(52) U.S. Cl. 435/6

(57) ABSTRACT

A method is provided for identifying in eukaryotes nucleic acid sequences that are involved in or suspected of being involved in cell growth regulation comprising mutagenising wild type plants or plants genetically modified to have accelerated tissue growth and selecting mutants having relative to the background and altered phenotype, followed by identifying the mutated gene and using said gene fro idantifying in a eikaryotic organism including a human a sequence involved in cell growth regulation e.g. tumour suppressors.

### FIELD OF INVENTION

The present invention relates generally to the field of identifying genes or functions of gene products in eukaryotic organisms. In particular, a novel method is provided that involves the use of a genetically modified plant model for identifying in a mammal including a human, nucleic acid sequences encoding products that are involved in the regulation of cell growth, including growth of cancer cells, in the mammal.

### TECHNICAL BACKGROUND

Important characteristics of mammalian cancer cells include their ability to invade and colonise neighbouring tissues and to proliferate in an uncontrolled manner at an abnormally high rate. High proliferation rates are caused by high cell division rates, which in turn are caused by defects in the regulatory systems that regulate the cell cycle. Thus, an uncontrolled cell cycle causes mammalian cells to change their programmed developmental destiny and to follow alternate differentiation routes leading to neoplastic transformations.

Whereas an upregulated cell cycle causing hyperplasia may lead to dysplasia and eventually tumours in mammalian subjects, similar upregulated cell cycling in plant cells leads to an abnormally increased amount of new cells that are differentiated and correctly positioned (Doerner et al. Nature 1996). Thus, it appears that plant cells are inherently capable of staying on a correct developmental pathway even though their cell cycle is out of control. This resistance to tumour growth is a remarkable feature of plant cells.

The eukaryotic cell cycle is regulated by complex mechanisms involving protein kinases, phosphatases and other proteins that regulate their interactions. The key components of this regulatory system are the CDK (Cyclin Dependent Kinase) complexes, which phosphorylate various components of the cell division machinery such as histones and lamins. A CDK complex consists generally of a catalytic unit, the CDK, and a regulatory unit, Cyclin, which is produced and degenerated at precisely defined points during the cell cycle. In a recent review by Vandepoele at al., The Plant Cell, 14:903-916 the presently known core cell cycle genes including CDKs and Cyclins in Arabidopsis thaliana and, to the extent known, their function have been described. It should be noted that the nomenclature in this review differs from that used herein, as the nomenclature as used herein is based on that used in the references herein.

When the study of plant cell cycle regulation was originally initiated, it was expected to find regulatory compounds similar to those found in other eukaryotes ranging from yeast to mammals. It was, however, also expected, that the cell cycle control in plants would reflect the differences between plant cells and mammalian cells. Such differences include: the presence of cell walls, a precise orientation of cell division planes and totipotency of already differentiated cells. It turned out that the central regulatory components of the cell cycle in plants, yeast species and animals are fairly similar (Hemerly et al. PNAS 1992, Mironov et al. Prog Cell Cycle Res 1997) and that in fact the cell cycle regulation in yeast, animals and plants may be regarded as variations of a common phenomenon.

In the extensively studied model plant, Arabidopsis thaliana, one of the major CDK complexes that regulates entry end exit from mitosis consists of a CDK kinase part termed Atcdc2a and a Cyclin part termed AtcycB. Similarly, type-D cyclins may combine with the Atcdc2a kinase to form the START kinase complex that initiates the exit from the G1-phase and entry into the S-phase of the cell cycle. The expression pattern of the Atcdc2a gene defines the cells that are competent for cell division, whereas the expression pattern of AtcycB defines the cells that actually undergo divisions. As it is shown herein, if the Atcdc2a promoter (prAtcdc2a) that directs this pattern is fused to the coding region of the AtcycB gene coding region and this gene construct is transformed into Arabidopsis thaliana plants, the AtcycB gene will be expressed in all cells that are competent to divide. This will result in plants that initiate cell cycles and development of new organs abnormally frequently and that also exhibit an abnormally rapid cell cycle. Plants that have been genetically modified such as those described above to have an increased cell cycle rate or growth are also referred to as being cell cycle stressed.

Genes that put a brake on the cell cycle, thus regulating it negatively, may be considered as tumour suppressor genes, as they allow the cells to undergo controlled proliferation and differentiation. Homologues of several tumour suppressor genes known from animals exist in plant cells. One notable example hereof is the RB gene coding for the retinoblastoma protein Rb which is part of a conserved pathway that controls the activation of cell division in animals in that it represses cell cycle transcription factors of the E2F family, and thereby prevents uncontrolled cell proliferation in animals [12, 13]. Thus, it has been found that the mammalian RB gene is frequently mutated in many tumours such as retinal tumours. The Rb protein shows a relatively high similarity between plants and mammals.

Accordingly, if tumour suppressor genes undergo “loss of function” mutations in animals it may lead to cancer. The same phenomenon may be hypothesised for plants. However, it is evident from analyses of hundreds of phenotypes from collections of randomly mutated plants that tumour associated phenotypes are virtually absent. As plant investigators agree that most, If not all, possible phenotypes in the model plant Arabidopsis thaliana have now been revealed, it would appear that mutations in putative tumour suppressor genes are either lethal or do not have any effect in plants. This may be because a defective tumour suppressor gene causes alternative phenotypes such as e.g. plants having higher proliferation rates that are difficult to detect, since this will result in higher growth rates which also are influenced by external factors.

The present invention relates to a novel and relatively simple method of discovering in eukaryotic organisms including mammals, genes involved in the regulation of cell growth, such as tumour suppressor genes, and the genetic code of such genes by using plants as a model system for this purpose. Alternative methods of identifying cell growth regulating genes (e.g. tumour suppressors) in animal model systems will be significantly more laborious and difficult to carry out.

The present inventor proposes that if mutations in putative tumour suppressor genes in plants do not have adverse effects on plants carrying such mutations, it is either because the cell cycle regulation of the plant adopts to the genetic changes in a different way than animal cells do or because the cell cycle regulation of the plant is more effective than that of mammalian cells. In both instances, if tumour suppressor gene candidates should be isolated from plants, the method of detecting such candidates had to be different from the common approaches that have been state of the art until today.

One possible alternative approach to solving the problem of finding ways to detect tumour suppressor genes in plants is to create a plant model system that has higher sensitivity towards changes in cell proliferation rates. In accordance with the present invention this may advantageously be done by using plants that are cell cycle stressed by continuous and/or enhanced production in the plants of components, the presence of which lead to initiation of the cell cycle. Such a component may e.g. be a cyclin that is the regulatory part of the cell cycle initiating CDK-complex.

The novel strategy presented in the present application will also give new insight into the mechanisms that control cancer e.g. in mammals and provide means of identifying and investigating components involved in cell growth, such as tumour suppressors, that have not yet been discovered using the conventionally used methods in this field.

### SUMMARY OF THE INVENTION

Accordingly, one primary objective of the present invention is to provide a method of identifying in a eukaryotic target organism a nucleic acid sequence encoding a product that is involved in or is suspected of being involved in cell growth regulation in said target organism. This method comprises the steps of:

(a) providing a wild type plant or a plant that is genetically modified to have tissue exhibiting, relative to the tissue of its non-genetically modified parent plant, accelerated growth,

(b) subjecting a multiplicity of the wild type plant or the genetically modified plant, or parts thereof to a mutagenisation treatment,

(c) selecting from the thus treated plants or parts thereof mutant plants having, relative to the wild type plant or the genetically modified plant of step (a), a phenotype characterised by an altered morphological structure or an altered colour,

(d) identifying in said selected mutant plants nucleic acid sequence(s) having a nucleic acid sequence which is different from the corresponding sequence(s) in the non-mutagenised wild type plant or genetically modified plant, and, using said nucleic acid sequence(s),

(e) identifying in the eukaryotic target organism a target nucleic acid sequence comprising a sequence encoding a product that is involved in cell growth regulation.

It is evident that upon identification of target nucleic acid sequences in a given eukaryotic organism including a mammal such as a human, the function of the genes and their products and hence their potential role in tumour cell growth suppression can be investigated further using methods which are either conventionally used methods or methods based on such conventional methods, but modified to fulfil specific requirements. This investigation may be carried out in plant cells as well as non-plant cells.

Based upon the above method of the invention it is also possible to provide the means for exploiting possibilities of using the nucleic acid sequence identified in the selected mutant plants and/or the gene product hereof in diagnosis, prophylaxis and/or treatment of cancer and also for exploiting the possibilities of using the identified eukaryotic target nucleic acid sequence (i.e. a sequence that is a homologue of the sequence identified in the mutant plant) or the polypeptide products hereof in diagnosis, prophylaxis and treatment of cancer. Accordingly, the use of any of such sequence(s) or gene products in the manufacturing of a medicament for treatment or prophylaxis of a cancer disease in a mammal, or the use hereof as a component in a diagnostic or prognostic kit are contemplated.

In presently preferred embodiments, the method for identifying and isolating the target (e.g. the mammalian) nucleic acid sequences which encode such protein products that potentially regulate cell growth, such as tumour suppressors, is based on the creation of a population of genetically modified plants including transgenic plants, which are modified to have an increased cell division frequency. This increased cell division frequency leads to a population of plants having an overall accelerated growth and development while typically retaining an overall morphological structure similar to a wild type plant from which they are derived. From this population a collection or “bank” of mutant plants having, relative to the genetically modified parent plants, a phenotype characterised by an altered morphological structure or an altered colour is generated. This change in phenotype of the mutant plants may be associated with “knock out” or activation of genes involved in the regulation of plant cell growth in either case resulting in a detectable altered phenotype as defined herein.

Although an altered phenotype in mutants plants may be recognised more readily or at a higher frequency when derived by subjecting genetically modified, i.e. cell cycle stressed plants as defined herein to mutagenisation than is the case if wild type plants are mutagenised, the latter approach may be appropriate such as in cases where mutant plant phenotypes derived from a cell cycle stressed, i.e. a genetically modified plant background are lethal or infertile. However, it should be noted that generally, mutant phenotypes are more readily detectable when cell cycle stressed plant backgrounds are used.

In further embodiments, both a genetically modified plant background and a wild type plant background is used for providing mutated plants showing the altered phenotype.

Subsequently, the mutations in the plants being phenotypically characterised by an altered morphological structure or colour relative to a non-mutagenised genetically modified plant are identified. As the altered morphological structure or colour of these mutant plants is the result of one or more genetic variations in genes controlling the development of the plants, these plants potentially harbour mutations in genes involved in cell cycle regulation including genes having tumour suppressor or suppressor-like activity.

Accordingly, such cell growth regulating gene candidates can subsequently be identified as the nucleic acid sequences present in the mutant plants that are functionally associated with the altered structure phenotype of the mutant plants. After the identification of affected cell cycle regulating genes e.g. genes having tumour suppressor activity, the nucleic acid can be isolated and used to identify wild type target analogues or homologues present in eukaryotic target organisms, e.g. plants, including the starting plant used in the present method, or mammals including humans thereby identifying cell growth regulators, such as tumour suppressors, in the target organism.

From this point, functional investigations of the eukaryotic gene analogues or homologues may be carried out to verify the exact role of the gene. When such cell growth regulator genes are identified and their exact function determined the possible uses of the gene homologues and their gene products in treatment of mammalian cancers can be exploited based upon conventional techniques.

Accordingly, the invention pertains in a further aspect to a method of determining the tumour suppressor activity, if any, of a gene product encoded by an eukaryotic cell gene and suspected of having tumour suppressor activity, the method comprising the steps of:

(a) providing a wild type plant or a plant that is genetically modified to have tissue exhibiting, relative to the tissue of its non-genetically modified parent plant, accelerated growth,

(b) subjecting a multiplicity of the wild type plant or the genetically modified plant, or parts thereof to a mutagenisation treatment,

(c) selecting from the thus treated plants or parts thereof a mutant plant having, relative to the wild type plant or the genetically modified plant of step (a), a phenotype characterised by an altered morphological structure or an altered colour,

(d) identifying is said selected mutant plant a nucleic acid sequence having a sequence which is different from the corresponding sequence in the non-mutagenised wild type plant or the genetically modified plant, and, using said different nucleic acid, identifying in the eukaryotic cell a homologue or analogue gene putatively involved in cell cycle regulation,

(e) transforming the coding sequence of said homologue or analogue gene into a mutant plant of step (c) under conditions permitting the sequence to be expressed, and

(f) determining whether or not the thus transformed mutant plant reverts to its wild type phenotype, such reversion being indicative of tumour suppressor activity of the homologue or analogue gene product.

### DETAILED DESCRIPTION OF THE INVENTION

In the first step of the present method of identifying in a eukaryotic target organism a nucleic acid sequence encoding a product that is involved in cell growth regulation in said target organism there is provided a wild type plant or a plant that is genetically modified so as to have tissue that, relative to the tissue of its non-genetically modified parent plant, exhibits accelerated growth.

As used herein, the expression “accelerated growth” is to be understood as at least one of the following characteristics (relative to the parent plant): (i) higher turnover rate of one or more meristemic cells, (ii) an increased proportion of rapidly dividing cells in a meristem, (iii) more rapid primordium formation, (iv) faster temporal initiation of organs such as flower formation at similar plant size leading to earlier flower formation.

As the parent plant may be used any monocotyledonous or dicotyledonous plant that can be genetically modified. Presently preferred parent plants include Lotus japonicus, Medicago truncatula, Oryza sativa, Antirrhinum majus and Arabidopsis thaliana.

A particularly useful parent plant for the present purpose is Arabidopsis thaliana including any ecotype hereof. This plant species is a widely used model plant whose genome has now been completely sequenced. Other advantages of using Arabidopsis thaliana include the ease with which genetic crosses and large amounts of biological material such as mutant plants and seeds can be made with this species. Thus, transformation of Arabidopsis thaliana is e.g. facilitated by the use of a very efficient and easy-to-use “flower dip” transformation procedure permitting more than a hundred individual transformants to be produced from one single parent plant within a few months time. Additionally, stable mutants of Arabidopsis thaliana can be produced using this transformation procedure e.g. in combination with transposon tagging systems that are readily available.

In the present context, the expression “genetically modified” refers to any modification in the wild type genome of the parent plant that leads to a detectable degree of accelerated growth in a tissue of the modified plant. In accordance with the invention “genetically modified” will include plants which, due to spontaneous mutation(s), have a detectable level of accelerated growth as well as plants that have been modified using recombinant DNA techniques to have accelerated growth in at least one tissue.

Any conventional techniques for modifying the genome of a plant can be used, such as the introduction into one or more cells of the parent plant of a nucleic acid sequence encoding a gene product that is actively involved in regulation of the cell cycle of the selected plant, representative examples of which are given in the following. Such coding sequences may be homologous or heterologous and they are inserted under conditions where they are expressed in the plant. Thus, the inserted coding sequence may be in the form of a construct comprising, in addition to the coding sequence, one or more nucleic acid sequences that permit(s) the coding sequence to be expressed such as e.g. a promoter sequence. Alternatively, a coding sequence may be inserted at a position where it becomes operably linked to such regulatory sequences present in the parent plant. The introduction into the parent plant of the coding sequence may conveniently be carried out using any conventionally used technique for introducing nucleic acid into a plant cell, e.g. transformation, electroporation or particle bombardment techniques.

As an alternative to providing a transgenically modified plant, the genetically modified plant can be provided by mutation e.g. mutations that inactivate genes coding for products that have an inhibitory effect on the plant cell cycle, or mutations that result in an enhanced expression of a gene product having a positive regulatory effect on any of the above characteristics of accelerated growth. Such mutations can be generated by any conventional mutagenesis procedure including e.g. chemical or UV mutagenesis or site directed mutagenesis e.g. using transposable elements.

In one useful embodiment, the accelerated growth of tissue of the genetically modified plant is caused by overexpression in the plant of a gene selected from the group consisting of a gene coding for a cyclin such as e.g. a gene encoding a cyclin B and a gene encoding a G1 cyclin, a gene coding for a transcription factor including E2F and myc, and any other gene positively affecting the cell cycle regulatory system including any of the cell cycle genes disclosed in Vandepoele et al. (2002) which is incorporated herein by reference, and homologues hereof isolated from any other organisms. It will be appreciated that such an overexpression can be provided by any conventionally used means for enhancing the expression of plant genes. One useful approach is to modify the plant by operably linking the coding sequence of the gene to a homologous or heterologous promoter having a stronger activity than the native promoter of the gene. Alternatively, the overexpression of the gene can be obtained by inactivating genes coding for gene products that inhibits the expression of the gene in the wild type plant or by introducing additional copies of a gene the product of which promotes cell cycle activity.

Thus, in a further useful embodiment of the invention, the genetically modified plant is obtained by introducing into a cell of the parent plant a gene construct comprising a promoter that directs high level expression in cell cycle active regions and, operably linked thereto, a nucleotide sequence encoding a gene product that is involved in acceleration of growth in a tissue of the thus modified plant such as a gene product that is capable of activating the cell cycle regulatory system of the plant.

The promoter of the gene construct is selected from any promoter that is capable of directing expression of a coding sequence in a plant cell. Thus, the promoter may be of prokaryotic or eukaryotic origin including a homologous or heterologous plant gene promoter which may be an inducible or a constitutive promoter. In the present context, useful plant gene promoters include promoters directing the expression of any cell cycle-associated plant gene including those disclosed in Vandepoele et al., supra, which is hereby incorporated by reference such as the Arabidopsis thaliana Atcdc2a promoter (prAtcdc2a) which is known to direct high-level expression in cells that are competent for cell division. Using such a promoter will limit the subsequent search for altered phenotypes to cell cycle active regions in the transgenic plant. Further examples of useful plant promoters include the Atcdc2b promoter (prAtcdc2b), also derived from Arabidopsis thaliana , and a 35S promoter from cauliflower mosaic virus.

In specific embodiments of the gene construct of the invention, the gene product is encoded by a gene selected from the group consisting of a gene coding for a cyclin including any of the genes disclosed in Vandepoele et al., supra, a gene coding for a transcription factor including E2F and myc, and any other gene positively affecting the cell cycle regulatory system. In the present context, particularly useful genes coding for a cyclin is selected from the cyc1At gene (encoding the mitotic cyclinB), the AtcycD2 gene (encoding a G1 cyclin) and the AtcycD1 gene, as these genes have shown to accelerate cell division frequencies when overexpressed in plants.

The above gene construct is advantageously provided with a polyadenylation site sequence. In specific embodiments the poly-adenylation site is derived from the Nopaline synthetase gene of Agrobacterium tumefaciens, an octopine synthetase gene or 35S poly-adenylation sequences.

The gene construct is introduced into the parent plant cell using any conventional means of introducing nucleic acid into plant cells such as those mentioned above. A particular useful means for introducing the gene construct is a transformation procedure using an Agrobacterium species such as Agrobacterium tumefaciens or Agrobacterium rhizogenez, a procedure that involves that the above gene construct is transferred to a plant transformation vector where it is flanked by DNA fragments carrying left and right borders for the Agrobacterium Transfer DNA (T-DNA). Upon Agrobacterium-directed transformation the complete construct will become integrated in the plant genome flanked by the T-DNA borders. Preferably, the transformation vector carries one or more selectable marker gene(s) allowing for selection of transgenic plants having one or more copies of the construct inserted. The transformation vector is introduced into cells of an Agrobacterium sp. such as A. tumefaciens or A. rhizogenez, which provide virulence factors in trans for subsequent plant transformation.

The thus obtained recombinant Agrobacterium cells are used to transform the selected parent plants by any of wounding, leaf disc or flower dip transformation procedures. Using Arabidopsis thaliana as the parent plant, the preferred transformation procedure is flower dip transformation.

In certain preferred embodiments of the invention, transgenic plants carrying the above gene construct carry a well-defined number of transgene copies. In order to produce such transgenic plants carrying a well-defined number of transgenes, two procedures may be used. For both of these procedures it is required to contain the gene construct in a plant transformation vector that carries a selectable marker. Also, it is required to end up with homozygous transgenic plants carrying two identical insertion copies of each of the various selectable marker constructs. In this way stable plants carrying e.g. two, four or six copies can be produced. This is achieved either by repeating the transformation procedure successively using plant transformation vectors e.g. carrying the Kanamycin, the Basta and the Hygromycin plant resistance genes, or by crossing the stable transgenic homozygous Kanamycin, Basta and Hygromycin resistant plants to double or triple homozygosity. These stable homozygotes will pass all their transgenes into every subsequent self-pollinated (Sn) generation.

In subsequent steps (b) and (c) of the present method, a multiplicity of the provided wild type plant or genetically modified plant, or parts thereof are subjected to a mutagenisation treatment, and from the thus treated plants or parts thereof mutant plants are selected having, relative to the wild type plant or genetically modified plant of step (a), a phenotype characterised by an altered morphological structure or an altered colour.

The expression “an altered morphological structure” is intended to mean any change in structural configurations of any part of the mutant plant that is not found in the parent plant backgrounds. Numerous altered phenotypes can be generated by mutation of wild type plants and cell cycle stressed plants. Thus, such phenotypes include production of extra organs, e.g. triple cotyledons, five or more petals, extra rosette leaves, super-branching; oversize plants due to hyperproliferation such as giant plants with respect to height, plants in which individual organs are significantly larger than in the background, plants; hyperproliferation in specific cell layers, e.g. in leaf epidermis leading to curled leaves, in upper leaf epidermis leading to backwardly curved leaves, In lower leaf epidermis leading to forwardly curved leaves, in root pericycle cells leading to hyper-initiation of lateral roots; destruction of endoreplication leading to undifferentiated structures such as simple, double, twisted or four-forked Trichomes, “siamese trichomes”, i.e. more trichomes from a single cell, Trichome-less leaves (“Glabra” leaves), curled leaves; undifferentiated structures on otherwise normal organs, e.g. transparent filamentous cells on cotyledons, hypocotyls, transition zone or roots; or severely undifferentiated growth directly from seed germination such as e.g. green, rapidly dividing undifferentiated callus-like cells including such cells showing some signs of differentiation.

In the literature it has been demonstrated that the tumor suppressor relevant SIAMESE trichome phenotype may be enhanced severely by introduction of an overexpressing mitotic cyclin gene. It may therefore be concluded that introduction of cell cycle stress upon the plants, severely affects the strengths of the tumor relevant phenotypes, thus making identification of the phenotypes considerably easier and in some cases crucial than by using wild type plants.

Other examples of structurally altered phenotypes in mutant plants of the invention include elongated or shortened leaves or stems, aberrant number of leaves, changes of leaf size or configuration, changes of the outer appearance of flowers, change of mean number of flower, etc.

As used herein the expression “an altered colour” refers to any change of the colour intensity, tone or hue of any outer surfaces or parts of such surfaces, including leaf surfaces, which render the mutated plants distinguishable from the non-mutated parents.

The genetically modified plants that are used as the genetic background for the mutagenesis step are preferably selected on the basis of the following considerations:

Due to transgene position effects, transgenic plants with similar number of transgenes may show differences in phenotypic aspects. A transgenic plant that is suitable as the genetic background for mutagenesis is selected on the basis of an apparent accelerated cell cycle. In addition, it is necessary to ensure that none of the transgenes have integrated into a functional gene, which may affect the following scoring of phenotypes. Inverse PCR or uneven PCR can, as it is described in the following examples, be used a as test procedure for that purpose, as these modified PCR techniques can be used for amplification of partly unknown DNA sequences. The presently preferred method is uneven PCR using primers covering sequences in the left or right T-DNA borders. Additionally, it may be confirmed that a transgenic plant contains only one independent transgene integration using genomic Southern blotting.

Additionally, the overall morphological structure of the parent plant should be substantially retained in the genetically modified plants to be mutagenised, although a useful genetically modified plant exhibits accelerated growth compared to the wild type parent plants. If required, an accelerated cell cycle (growth) may be confirmed by determining the RNA level in the modified plant and comparing it with that of the wild type or by a cross to transgenic plants containing a construct of a cyc1At promoter fused to a degradable GUS reporter gene [9]. A more frequent number of blue-stained meristem cells evidenced by an appropriate GUS activity assay in such double transgenes will indicate a higher rate of cell division initiation.

Having selected appropriate wild type plants or genetically modified, e.g. transgenic, plants for the mutagenesis step, the mutagenisation is carried out using any conventional procedure which are well-known to those of skill in the art, such as e.g. EMS mutagenesis or any other chemical mutagenisation, UV mutagenesis, T-DNA-mutagenesis and mutagenesis using a transposable element. As target tissues for the mutagenisation treatment may be used whole plants or parts hereof such as leaves, stems, flowers or seeds. When seeds of genetically modified plants are subjected to mutagenisation, they can be grown to become M1 plants, which are heterozygous for possible mutations, thus not showing any evident altered phenotypes. Seeds from such M1 plants are M2 seeds in the sense that they germinate into M2 plants from which altered phenotypes may be identified. Thus, a mutant collection may be kept as a “bank of M2 seeds” and used for subsequent screening for any altered phenotypes.

In the subsequent step (c) mutant plants having, relative to the wild type plant background or the genetically modified plant background subjected to the above mutagenisation treatment, a phenotype characterised by an altered morphological structure or an altered colour are selected. However, the mutant bank will consist of a collection of heterozygous, mutated and transgenically modified cell cycle stressed plants that are homozygous or heterozygous with respect to the caused mutation. In order to reveal phenotypic changes and to map the mutated genes it may be necessary to produce an S1 self-pollinated generation from each of the plants. Any relevant altered phenotype should be scored from this population of homozygous plants.

The selection of mutant plants exhibiting a phenotype characterised by an altered morphological structure or colour is conveniently carried out on seedling plants, but can also be carried out using plants at a later growth stage. Seedlings showing. tumour formation or indeterminate meristematic growth are obvious phenotypes as defined herein, but the primary target may not necessarily be tumouric regions in the plants. E.g., if a plant harbours a genetic defect and as a result of that produces an abnormal number of lateral roots it may be comparable to an animal harbouring a corresponding genetic effect that produces tumours.

In order to identify plant phenotypes that are likely to correspond to animal tumour phenotypes, an RNA-inhibition approach may be used on either wild type plants or cell cycle stressed, i.e. genetically modified plants to produce plant model phenotypes that can be used as the reference phenotypes against which mutant plants having an altered phenotype as defined herein can be selected.

For such an approach, a set of vectors that promote the production of loss-of-function plants[10] by reverse genetics may be used [11]. The vectors contain a plant recognisable intervening sequence (intron) flanked by unique cloning sites. Two identical PCR fragments covering about 400 bp of the relevant cDNA are cloned in opposite directions, sense and antisense, on each side of the intron sequence. A suitable promoter, e.g. the 35S constitutive promoter directs expression of the sense-PCR-fragment:intron:antisense-PCR-fragment sequences. The complete construct is transferred to any plant transformation vector flanked by T-DNA borders, and subsequently transformed into plants. Transgenic plants are selected by the marker gene of the plant transformation vector. This causes the production of a double stranded RNA (dRNA) in the plant. The native plant defence system produces a defence against this dRNA. This defence response also targets the corresponding endogenous RNA, subsequently leading to the elimination of the desired gene product. The plants are crossed to homozygosity and analysed for phenotypic differences.

Thus, by using this so-called RNA inhibition (RNAi) system it is possible to eliminate the functions of known tumour suppressor gene homologues in plants. In this context, an alternative approach is to cross to plants having a mutation in a known tumour suppressor gene so as to test the mutation in a sensitive plant that might thereby produce an altered phenotype. As one example, the Retinoblastoma plant homologue (RBR) gene in Arabidopsis [12, 13] may be used to knock out the Retinoblastoma gene function. This may result in tumour cells in the plants, but could also lead to a different response in the plant such as for example elongated leaves. In this case an elongated leaf phenotype corresponds to a tumour phenotype in animals. In this way, a series of model plant phenotypes can be produced using homologues genes of known animal tumour suppressers or inhibitors of cell division. The phenotypes will represent defects in different signal transduction pathways in the plants and thus by analogy in animals.

In the selection step (c) of the present method, both wild type plants and cell cycle stressed (i.e. genetically modified) plants should be subjected to the above RNAi procedure to provide reference or model phenotypes against which mutant plants having, relative to the model phenotypes, an altered phenotype can be selected, as the difference in phenotypes of plants with these different genetic backgrounds serves as a proof of concept for the effect of adding a cell cycle stress factor to the plants. Thus, by using this procedure, a series or collection of relevant mutant phenotypes can be selected.

In the subsequent step (d) of the present method nucleic acid sequence(s) having a nucleic acid sequence which is different from the corresponding sequence(s) in the non-mutagenised genetically modified plant is/are identified in the selected mutant plants. In preferred embodiments, the nucleic acid sequence thus identified in step (d) and/or the product encoded by the sequence is functionally associated with the phenotype of the selected mutant plants of step (c).

Accordingly, in another embodiment of the invention, the identification of nucleic acid sequence(s) having a sequence which is different from the corresponding sequence(s) in the non-mutagenised genetically modified or transgenic plant is performed using a method selected from the group consisting of an Amplified Fragment Length Polymorphism (AFLP) method, a Single Sequence Length Polymorphism (SSLP), a differential display method, a restriction fragment length polymorphism (RFLP) method, a Single Strand Conformation Polymorphism (SSCP) method, allele specific amplification, restriction PCR, PCR, sequencing and a Single Nucleotide Polymorphism (SNP) method. Among these techniques, the SSLP technique is particularly preferred, optionally combined with the SNP method.

As an example, mutated genes of each selected phenotype from the EMS mutant collection are mapped using AFLP [14]. The Arabidopsis Landsberg erecta (Ler) ecotype or any Arabidopsis ecotype besides Colombia (Col-O) may be used for this mapping [15] (in the mapping an Arabidopsis ecotype different from that used for providing the mutant collection should be used irrespective of the mapping technique used). During this entire procedure it is important to consider that the genetic background contains additional gene constructs that will differ from the original Colombia genetic background. Accordingly, the cell cycle stressed genetically modified/transgenic plants, and not Col-O wild type plants, should be considered parent plants and thus included as control samples in these experiments which are carried out as follows:

Homozygous phenotypes are crossed with Landsberg erecta plants. The resulting F1 generation is self-crossed and will segregate 3:1. Homozygous phenotypes can now be subjected to AFLP tests in a bulk analysis where approximately 10 individual plants are collected in each bulk. Typically, AFLP is performed using a “rare primer” (preferably containing the EcoRI site) and a “frequent primer” (preferably containing the MseI site as the recognition sequence for this enzyme is rich in As and Ts as in most plant genomes). In the first amplification round there should preferably be an addition of only one nucleotide (compared to the adapters) resulting in a 16-fold reduction of amplified products. In the second round of amplification there may be an addition of two nucleotides in each primer, alternatively a 2+3 addition resulting in further reductions. In this second round, the primers are phosphorylated with radioactive dATP. The fragments are analysed on high-resolution polyacrylamide gels. Markers found in this way may either be fragments absent in Ler parent or fragments of changed sizes. Markers that follow single phenotypes are compared to the general Col-0 wild type map [16] and the approximate location of the mutation in the Arabidopsis genome may in this way be deduced. The mutated locus is narrowed down and subsequently identified, either by complementation with TAC clones and/or sequencing of PCR fragments spanning the delimited region.

Having identified in the selected mutant plants of step (c) nucleic acid sequence(s) having a nucleic acid sequence that is different from the corresponding sequence(s) in the non-mutagenised wild type plant or the genetically modified/transgenic plant and that is/are therefore putatively associated with the regulation of the plant cell cycle, such sequences can be used for identifying in a eukaryotic target organism a target nucleic sequence comprising a sequence encoding a product that is putatively involved in cell growth regulation.

The eukaryotic target organism is selected from any eukaryotic organism including a microbial cell such as a yeast cell, a plant and an animal including invertebrates and vertebrates including mammals, birds, fish, amphibia, reptiles and insects. A mammalian target organism may be selected from mice, rats, guinea pigs, cats, dogs, apes, and primates including humans.

In one useful embodiment, the target nucleic acid sequence is identified in step (e) of the present method by carrying out a homology search in a genome database for the selected target organism or by molecular probing which using e.g. a method selected from PCR, Northern blot, Southern blot, arraying and direct sequencing.

As commonly defined (see e.g. Encyclopaedia of Life Sciences/www.els.net, Nature Publishing Group, 2000) homology is defined herein as sequence identity between genes or proteins at the nucleotide or amino acid level, respectively.

Thus, in the present context “protein homology” is perceived as a measure of sequence identity or sequence similarity between proteins at the amino acid level and may be determined by comparing the amino acid sequence in a given position in each sequence when the sequences are aligned. Thus, as used herein “protein homology” may refer to amino acid sequence identity of at least 20%, preferably at least 30% identity e.g. at least 40% identity or at least 50% or at least 60% identity. At the nucleotide sequence level homology may be defined as a sequence identity that is at least 20%, preferably at least 30%, e.g. at least 40%, including at least 50% such as at least 60% or at least 70%.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity # of identical positions/total # of positions (e.g., overlapping positions)×100). In one embodiment the two sequences are of the same length.

One may manually align the sequences and count the number of identical amino acids. Alternatively, alignment of two sequences for the determination of percent identity can be accomplished using a mathematical algorithm. A preferred, non-limiting example of a mathematical algorithm utilised for the comparison of two sequences is the algorithm of Karlin and Altschul (1990) Proc. Natl. Acad Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) ). Mol. Biol. 215:403-410. BLAST nucleotide searches can be performed with the NBLAST program, score=100, word length=12, to obtain nucleotide sequences homologous to a nucleic acid molecules of the invention. BLAST protein searches can be performed with the XBLAST program, score=50, word length=3 to obtain amino acid sequences homologous to a protein molecule of the invention. To obtain gapped alignments for comparison purposes, Gapped BLAST can be utilised as described in Altschul et al. (1997) Nucleic Acids Res. 25:3389-402. Alternatively, PSI-Blast can be used to perform an iterated search, which detects distant relationships between molecules. When utilising the NBLAST, XBLAST, and Gapped BLAST programs, the default parameters of the respective programs, can be used. See http://www.ncbi.nlm.nih.gov. Alternatively, sequence identity can be calculated after the sequences have been aligned e.g. by the program of Pearson W. R and D. J. Lipman (Proc Natl Acad Sd USA 85:2444-2448, 1998) in the EMBL database (www.ncbi.nlm.gov/cgi-bin/BLAST). Generally, the default settings with respect to e.g. “scoring matrix” and “gap penalty” can be used for alignment. In the present application the BLASTN and PSI BLAST default settings was used.

The percent identity between two sequences can be determined using techniques similar to those described above, with or without allowing gaps. In calculating percent identity, only exact matches are counted.

In a further embodiment, the present method comprises a further step of isolating the target nucleic acid sequence identified in step (e).

Having identified the target nucleic acid sequence, any product encoded by the sequence may be derived and its function determined using any conventional methods for that purpose. It is contemplated that the product of a target nucleic acid sequence may be functionally active in a signal transduction cascade leading to suppression of cell growth in the target organism or it may be a suppressor of cell growth in the target organism.

The functional association between the plant nucleic acid sequence of step (d) and the target nucleic sequence that is identified using the present method can be assessed using several approaches. Thus, in one embodiment, a putative functional association between the plant nucleic acid sequence identified in step (d) and the target nucleic acid sequence is determined by homology analysis between said plant nucleic sequence and said target nucleic sequence. In further embodiments, such a putative functional association between the plant nucleic acid sequence identified in step (d) and the target nucleic acid sequence is determined by analysing the effect of expressing the target nucleic acid sequence in an in vitro model or an in vivo model for assaying cell growth regulation activity.

As mentioned above, one major objective of the present invention is to identify genes in mammals that are involved in cell growth regulation, in particular such genes coding for proteins having tumour suppressor activity. That such an objective has been met is illustrated by the above experiments showing that the Retinoblastoma gene in plants can be identified using the RNAi approach and as a consequence of the homology with mammalian homologues of this plant genes, it is evident that it will be possible, using the plant gene to identify the corresponding mammalian homologue.

In this context, further evidence is provided by the following findings: A mutant identical to the Arabidopsis pinoid mutant was selected in a cell cycle stressed mutant bank collection. This mutant exhibits production of extra organs (three cotyledons, five petals). The pinoid gene is a serine threonine kinase that promotes primordium development by negatively regulating auxin signalling. Onset of primordium development requires controlled growth and onset of differentiation. The primary role of a tumour suppressor is to control these two actions. Thus, as auxin induces transcription of mitotic cyclins and as several serine threonine kinases are involved in cell cycle regulation and since primordium promotion may be regarded as a link between crude cell cyclin and differentiation, the pinold-identical mutant gene constitute an evident cell cycle directed tumour suppressor candidate. This is also true if the mutant phenotype is caused by a mutation in another gene in the pinoid signalling pathway. These results indicate that the method of the invention is a useful biological model system for identifying cell cycle related tumour suppressor candidate genes.

A still further useful example of a useful model for screening for tumour relevant mutants in plants is the use of so-called endoreduplication mutants for such a purpose. The endoreduplication process is a well-described variation of the traditional cell cycle, which confers specific differentiation programmes upon some cells in higher eukaryotes. In plants, the endureduplication cycle directs the proper development of the leaf epidermis, especially the development of trichome cells in the epidermis. Destruction of the endoreduplication cycle may lead to hyperplasia in the epidermis layer, cell death, or siamese trichomes. Thus leaf epidermis, and especially trichome developmental mutant may be indicative of endoreduplication disruption.

The relevance of the endoreduplication cycle with respect to tumour formation is due to the fact that mitosis has to be inhibited in order to perform the endoreduplication cycle variant. Mitotic inhibitors are per definition tumour suppressing inhibitors, as most types of mammalian tumours show hyper-frequency mitosis. Thus a mitosis-directed controller of endoreduplication in plants may function as a tumour suppressor in mammals. In a mutant bank created using the method of the invention the mitotic Arabidopsis cyclin AtcycB1.1 is used as a cell cycle stress factor. Thus mutations in genes encoding inhibitors of this mitotic cyclin may account for the high number of trichome mutants (1-2%) found in this screening system.

In a further aspect of the invention there is provided a method of determining the tumour suppressor activity, if any, of a gene product encoded by a eukaryotic cell gene and suspected of having tumour suppressor activity, the method of determining the tumour suppressor activity, If any, of a gene product encoded by an eukaryotic cell gene and suspected of having tumour suppressor activity, the method comprising the steps of:

(a) providing a wild type plant or a plant that is genetically modified to have tissue exhibiting, relative to the tissue of its non-genetically modified parent plant, accelerated growth,

(b) subjecting a multiplicity of the wild type plant or the genetically modified plant or parts thereof to a mutagenisation treatment,

(c) selecting from the thus treated plants or parts thereof a mutant plant having, relative to the wild type plant or the genetically modified plant of step (a), a phenotype characterised by an altered morphological structure or an altered colour,

(d) identifying is said selected mutant plant a nucleic acid sequence having a sequence which is different from the corresponding sequence in the non-mutagenised wild type plant or the genetically modified plant, and, using said different nucleic acid, identifying in the eukaryotic cell a homologue or analogue gene putatively involved in cell cycle regulation,

(e) transforming the coding sequence of said homologue or analogue gene into a mutant plant of step (c) under conditions permitting the sequence to be expressed, and

(f) determining whether or not the thus transformed mutant plant reverts to its wild type phenotype, such reversion being indicative of tumour suppressor activity of the homologue or analogue gene product.

It should be understood that for any of the above steps which is equivalent to the steps of the above method of identifying a nucleic acid sequence encoding a product that is involved in or is suspected of being involved in cell growth regulation in a target organims as defined herein, corresponding embodiments and modifications can envisoned.

The person of skill in the art will appreciate that any genes or gene products identified using the methods of the invention are of potential use in diagnosis, prophylaxis and treatment in cancer diseases and other diseases characterised in abnormal cell growth patterns. Thus, the use of such genes and gene products as diagnostic agents and as medicaments for preventing and treating disease conditions such as cancer is contemplated.

### EXAMPLES

The invention will now be illustrated in the following, non-limiting examples.

### Example 1

Construction of AtcycD2 Overexpressing Arabidopsis thaliana Plants

1.1. Construction of a Gene Construct Comprising the AtcycD2 Gene Linked to a Plant Promoter

The Promoter Region

A 1.7 kb promoter fragment was isolated using the following PCR protocol:

Standard PCR Reaction

(Unless stated otherwise this was the standard PCR reaction in all the following descriptions):

10 ng DNA

5 pmol of each primer

mM dNTP (Sigma)

1×PCR buffer (Sigma)

0.2 units Taq Polymerase (Sigma)

Total vol of 25 μL

36 cycles of: 94° C.—45 sec, 50° C.—45 sec, 72° C.—3 min.

The 5′ start site was randomly chosen. A SalI site was introduced in the 5′-PCR primer (AAGTTTTGTCGACATATATAT) (SEQ ID NO:1) as cloning site. Similarly, a NcoI site was introduced as cloning site in the 3′-PCR primer (AACCTGATCCATGGATTCCTG) (SEQ ID NO:2) spanning the promoter fragment to the ATG start site of the Atcdc2a gene. (NcoI recognition site contains the ATG).

The Coding Region

The coding region of the AtcycD2 gene was isolated from an Arabidopsis thaliana poly-dT primed cDNA seedling library, using a genomic PCR fragment:

| | |
|---|---|
| ATGGCTGAGA ATCTTGCTTG TGGTGAAACC | (SEQ ID NO: 3) |
| AGCGAGTCAT GGATCATTGA CAACGACGAT | |
| GATGATATCA ACTATGGCGG CGGATTTACG | |
| AACGAGATTG ATTACAATCA CCAACTTTTT | |
| GCTAAAGACG ACAACTTTGG CGGCAACGGA | |
| TCAATTCCGA TGATGGGTTC TTCTTCATCG | |
| TCCTTGAGTG AAGACAGAAT CAAAGAGATG | |
| TTGGTGAGAG AGATTGAGTT TTGCCCTGGA | |
| ACTGATTATG TTAAGAGATT GCTTTCTGGT | |
| GATTTGGATT TGTCTGTTCG AAACCAAGCT | |
| CTTGATTGGA TTCTAAAGGT ATGTGATTTC | |
| TTCTTTTGAT TGCTTTGATC TGTGTTGTGT | |
| TTTAAGACAA ACCTCAAATC TGGTTTGGTC | |
| TCTTTTTGTG CTTTGTGATT GAGTTTAAGT | |
| GTGTTCTTTG ATCAGTTTGA TAGCTCATTC | |
| ATTCTCTGAT GTATTTATTA TAGATTAATT | |
| GAATCTTCTC TATAGATTGA GGTATTGATC | |
| TTGTTGAGTA ATTGAGTTCT TTTGTCTTTA | |
| ATGCCAATTG TTCTACAGGT TTGTGCTCAT | |
| TACCATTTTG GACATCTGTG CATATGCCTA | |
| TCCATGAACT ACTTGGATCG GTTCTTAACA | |
| TCCTATGAAT TGCCG | |

spanning the 5′ coding region as a probe. A full length AtcycD2 cDNA clone was isolated and zapped into a pBluescript subclone.
Fusion of Regions

A NcoI site perfectly positioned covering the ATG start site of the AcycD2 cDNA allowed the fusion of the Atcdc2a promoter with the AtcycD2 coding region in the pBluescript/AtcycD2 subclone. The SalI site was used as the 5′ cloning site.

Adding a Poly-Adenylation Site

The nopaline synthetase poly-Adenylation site was added to the fusion construct by inserting the PrAtcdc2a-AtcycD2 fragment into the pznAn plasmid (a pzp plasmid derivative carrying the Nopaline synthetase promoter in front of the Kanamycin selection marker and containing the nopaline synthetase poly-Adenylation site in the polylinker). The prArcdc2a-cyc1At fusion was introduced in front of the pAnos using SalI as the 5′ cloning site. A SacI site positioned in the 3′-UTR in front of the polyAdenylation site of AtcycD2 was used as the 3′ cloning site, positioning the pAnos 3′ of the AtcycD2 coding region.

### Example 2

The construction and characterisation of a genetically modified Arabidopsis thaliana plant

Plant Transformation Vectors

The prAtcdc2a-AtcycD2-pAnos construct of Example 1 was transferred to the plasmids of the pGreen series of plant transformation vectors [17] carrying either the plant bar gene (Basta resistance) or the Hyg gene (Hygromycin resistance) using SalI:ApaI subclonings. The clones were selected on Kanamycin (25 μg/μL) plates

Agrobacterium Transformation

The resulting prAtcdc2a-AtcycD2-pAnos construct was electro-transformed into the super-virulent RecA Agrobacterium strain AGL1. Transformant cells were selected by Rifampicin (100 μg/μL) and Kanamycin (100 μg/μL) resistance.

Arabidopsis Transformation

Flowering Arabidopsis thaliana plants of the Colombia ecotype Col-0 was transformed using the above transformed cells by floral dipping [18].

20 Col-0 seeds were sown in soil in 10×10 cm pots. Flowering plants were cut down once and plants with a second set of flowers were used for transformation.

A single colony of the transformed Agrobacterium strain was freshly plated on a selective plate. After two days a bacterial scrape was used to inoculate 300 mL of LB-medium containing the appropriate antibiotics. Cells were grown to log phase (OD1.2-1.5) and harvested at 6,000×g for 15 minutes. Cells were resuspended in a solution of 10 mM MgSo4 and 5% sucrose and transferred to a 300-mL beaker. Silwet L77 “Vac in Stuff” was added to the solution just prior to use. Arabidopsis flowers were submerged into the solution for 10 sec to 1 minute. The plants were enclosed in transparent plastic bags and left overnight at 23° C. under a diurnal growth light source to allow the bacteria to drip off. On the following day the bags were opened to expose the plants to less humidity. After one more day, bags were removed and the plants were left to grow in growth-chambers with a diurnal light rhythm. As siliques began to form and brownish, the plants were transferred into A4 envelopes, pre-sealed at the bottom with tape, preventing seed spill. The envelopes were left at room temperature under dry conditions for one week. Seeds were harvested by squeezing the envelopes and subsequently sieved. Seeds were stored under dry conditions at room temperature.

Selection of Transgenic Plants

About 10,000 seeds were rinsed for one minute in 70% EtOH sterilised in Na-Hypochlorate containing a few drops of Tween 20 for 20 minutes. The seeds were rinsed 5× in sterile water and spread onto selective plates supplemented with ampicillin 50 μg/mL and (Hygromycin 30 μg/mL or Basta 100 μg/μL or Kanamycin (50 μg/ml) in MS (Murashige-Skoog) substrate, 1% sucrose, 1% agar noble) plates. Resistant seedlings were transferred to Leca pearls as primary transformants and allowed to set seeds.

Selection of Stable Homozygous Transgenic Plants

About 50 seeds from each plant of this S1 generation were screened for single or multiple T-DNA insertions on relevant selective plates. Single insertions resulted in a 3:1 segregation ratio, whereas multiple insertions resulted in an excessive numbers of resistant seedlings. Resistant plants from single insertion lines were self-crossed and the seeds screened once again. 100% resistant plants originate from homozygous lines. The homozygous lines were saved and phenotypically analysed by various means as considered above. Finally each homozygous S1 plant was screened for insertion position of the T-DNA.

Screening for T-DNA Insertion Positions

The position of a T-DNA in the Arabidopsis genome was identified by uneven PCR. Only one of the primers in each round of PCR was specific, the other primer was a mix of 10-mers with an arbitrary sequence, which would anneal to several sites on the template. If the distance between the specific primer and the 10-mer was less than 3-4 kb, the sequence between them was amplifiable. To avoid too many unspecific products, two different annealing temperatures were used in consecutive cycles of amplification.

The uneven PCR was done using 12 different random 10-mer oligonucleotides, and the specific primers and the nested primers covered either the left or the right border of the T-DNA. The buffer conditions and final volume were as in an standard PCR, the final concentrations of other ingredients were as follows: specific primer 0,27 pmol/μL, 10-mer 0,05 pmol/μL, dNTPs 0,22 mM, and 0,19 units of Taq polymerase.

Uneven PCR Protocol:

| | | |
|---|---|---|
| Temperature | Time | #cycles |

| | | | |
|---|---|---|---|
| 94° C. | 1 | min | 1 |
| 94° C. | 30 | sec | 3 |
| 55° C. | 1 | min | |
| 72° C. | 1 | min | |
| 94° C. | 15 | sec | |
| 42° C. | 1 | min | |
| 72° C. | 1 | min | |
| 94° C. | 15 | sec | 20 |
| 57° C. | 30 | sec | |
| 72° C. | 30 | sec | |
| 94° C. | 15 | sec | |
| 45° C. | 30 | sec | |
| 72° C. | 30 | sec | |
| 72° C. | 5 | min | 1 |

A second round of PCR was done on 0,5 μL of first round product with a nested specific primer and the 10-mer to further increase the amount of the specific product.

Uneven Nested PCR Protocol:

| | | |
|---|---|---|
| Temperature | Time | #cycles |

| | | | |
|---|---|---|---|
| 94° C. | 1 | min | 1 |
| 94° C. | 15 | sec | 20 |
| 57° C. | 30 | sec | |
| 72° C. | 30 | sec | |
| 94° C. | 15 | sec | |
| 45° C. | 30 | sec | |
| 72° C. | 30 | sec | |
| 72° C. | 5 | min | 1 |

The PCR fragments were run on 1% agarose gets and specific bands were eluted, subcloned and sequenced. T-DNA insertions outside gene coding regions and putative promoter regions were selected for further studies.

### Example 3

Silencing of Putative Tumour Suppressor Genes in Arabidopsis

RNA Silencing of Retinoblastoma-Related (RBR) Gene in Arabidopsis Col-O Plants

Prior to subjecting the genetically modified (cell cycle stressed) plants of Example 2 to mutagenesis, selected transgenic cell cycle stressed plants were tested for silencing of various putative tumour suppressor genes. In this example the silencing of the for the RBR gene is described:

Four PCR primers flanking a central intron-free region of the Arabidopsis Retinoblastoma related gene, RBR, was produced.

An antisense PCR-fragment was synthesised using primers flanked by a BamHI recognition site: cgcggatccgaagagcacacataatattggaagc (SEQ ID NO:4) and a ClaI recognition site: CCATCGATCAGATGTCGTCTCAACTCTCTAGGC (SEQ ID NO:5).

Likewise a sense PCR-fragment was synthesised using primers flanked by KpnI recognition sites: CGGGGTACCAGATGTCGTCTCAACTCTCTAGGC (SEQ ID NO:6) and a XhoI recognition site: GCCGCTCGAGcgaagagcacacataatattggaagc (SEQ ID NO:7).

The BamHI-ClaI antisense PCR fragments were digested with the relevant restriction enzymes and sub-cloned into the pKannibal vector. Positive clones were Kanamycin resistant pKan-BC clones. The KpnI-XhoI sense PCR fragment was eventually subcloned into the pKan-BC constructs resulting in a pKan-BCXK construct containing an approximately 800 bp intron sequence flanked by the BamI-ClaI antisense fragment on one side and the KpnI-XhoI-sense fragment on the other. This central part of the construct was transferred into the NotI site of the plant transformation vector pART for transformation into the Agrobacterium strain AGL1 and subsequently transformed into Arabidopsis Col-0 and Arabidopsis cell cycle stressed transgenic plants, respectively.

Homozygous plants of both types of transformed Aradopsis plants were selected as described above and analysed and compared for phenotypic differences. The resulting plants served as model reference phenotypes in the subsequent screening for phenotypes in the EMS mutant plant bank produced in Example 4.

### Example 4

Construction of a Mutant Plant Bank

EMS Mutagenesis of Arabidopsis

The mutagenisation was performed according to the method described by Steve Jacobsen on http://www.mcdb.ucla.edu/Research/Jacobsen/EMSmutagenesis.html as follows:

0.2 gram (approximately 10,000) of mutant background seeds obtained from a transformed (cell cycle stressed) Arabidopsis plant obtained in Example 2 was washed in 0.1% Tween for 15 minutes and transferred to 15 mL ddH2O. 15-45 μL of (0.1% to 0.3%) EMS was added. The mixture was incubated and rotated for 8-12 hrs in a fume hood. The EMS was removed and neutralised in 0.5 M NaOH overnight. Seeds were rinsed once briefly in ddH2O and then for 2-4 hrs in another 10 mL of ddH2O. Seeds were transferred into 100 mL of 0.1% Agar noble and sown in 100 pots using a P1000 Pipetteman.

Each mutant bank should end up containing approximately 1500 individual plants. Plants grow in greenhouses and seeds from the plants were collected for phenotypic screening.

Putative Phenotypes

Homozygous putative phenotypes, i.e. mutant plants showing a phenotype characterised by an altered morphological structure as compared to that of the parent transformed plant, were crossed to Arabidopsis Ler ecotype. From the resulting F2 population about 2,000 seeds were harvested. DNA was prepared from individual seedlings, bulked and used for Amplified Fragment Length Polymorphism (AFLP) mapping analysis as described below.

AFLP Mapping of Mutated Genes

In the AFLP-PCR reactions two sets of primers corresponding to EcoRI and MseI adaptors are used. The primers are provided with various numbers of additional nucleotides in the 3′end. The sequence of the basic primers were:

EcoRI adaptor primer: GATGAGTCCTGAGTAA (SEQ ID NO:8)+extra nucleotides MseI adaptor primer: GACTGCGTACCAATTC (SEQ ID NO:9)+extra nucleotides

Two single stranded complementary oligonucleotides named forward EcoRI adapter (5 pmol/μL) and reverse MseI adapter (50 pmol/μL) were mixed in equal amounts in order to create a double stranded adapter upon annealing. The reactions were incubated for 2 min at 80° C. and left to stand until room temperature was reached.

Genomic DNA from the relevant phenotype, i.e. the putative phenotype as defined above, was digested with EcoRI and MseI yielding DNA fragments of varying lengths. Subsequently, the annealed adapters were ligated to the DNA fragments in a total of 50 μL H₂0: 0.5 μg genomic DNA, 100 nM EcoRI pre-annealed adapter, 1 μM mess pre-annealed adapter, 10 μL 5×OPA buffer (5×One-Phor-All stock (Pharmacia), 0.25 mg/mL bovine serum albumin, 25 mM DTT), 200 μM ATP, 0,2 unit T4 DNA ligase, 5 units MseI and 10 units EcoRI.

This reaction was incubated 4 h at 37° C. and then diluted 5 times with TE_{0.1}.

In the pre-amplification round the following components were mixed in a total volume of 20 μL: 5 μL template DNA (above listed reaction), 50 ng EcoRI primer (one selective nucleotide), 50 ng MseI primer (one selective nucleotide), 0.25 mM dNTPs, 2 μL 10× Taq buffer, 0.5 unit Taq polymerase and H₂0 to 20 μL.

PCR were performed using the following cycle conditions: 20 cycles of: 94° C. for 30 sec., 56° C. for 1 min., 72° C. for 1 min.

5 μL of the pre-amplification reaction was analysed on a 1,5% agarose gel and if a faint smear was visible (as an indication of successful pre-amplification) the reaction was diluted 5 times with TE_{0.1}.

The reactions were labelled with ³P-ATP in a total volume of 0.5 μl 5 ng EcoRI primer (three selective nucleotides), 1.0 μCi 3P-ATP, 0.1 unit T4 polynucleotide Kinase, 0.05 μL 10×T4 polynucleotide Kinase buffer.

The reaction was incubated for 45 min. at 37° C. and placed on ice before use.

AFLP Second PCR Reaction

In a total volume of 20 μL the following components are mixed: 5 μL template DNA (diluted pre-amplification reaction), 30 ng MseI primer (three selective nucleotides), 0.5 μL labelling mix (above reaction), 0.25 mM dNTPs, 2 μL 10× Taq buffer, 0.5 unit Taq polymerase and H20 to 20 μL.

PCR was performed using the following cycle conditions:

12 cycles of:
94° C. for 30 sec.
65 down to 56° C. for 30 sec. (ΔT=0.7° C./pr. cycle)
72° C. for 1 min.

18 cycles of:
94° C. for 30 sec.
56° C. for 30 sec.
72° C. for 1 min.

7 μL of stop solution (Stop solution: 10 mM EDTA, 98% Formamide, 0.06% (w/v) bromo-phenol blue and 0.06%, xylene cyanol was added and the reaction denatured 3 min. at 90° C. and cooled on ice before analysis on a 6% polyacrylamide gel. The gel was autoradiographed with high speed X-ray film in a film cassette at room temperature and developed within 2-4 days.

Cloning of Isolated Markers

The selected band was excised from the dry 6% acrylamide gel and incubated ON at 37° C. in 100 μL Crush and Soak elution buffer (0.5 M ammonium acetate, 10 mM magnesium acetate, 1 mM EDTA (pH 8.0) and 0.1% SDS) The filter paper and gel waste was removed by centrifugation for 1 min at 4° C. Supernatant 1 was transferred to a new eppendorf tube and 50 μL CS buffer was added to the remaining gel fragment and paper. The mixture was

### REFERENCES

1. Cockcroft, C. E., et al., Cyclin D control of growth rate in plants. Nature, 2000. 405(6786): p. 575-579

2. Hemerly, A., et al., Genes regulating the plant cell cycle: isolation of a mitotic-like cyclin from Arabidopsis thaliana . Proc Natl Acad Sci USA, 1992. 89(8): p. 3295-3299

3. Mironov, V., M. Van Montagu, and D. Inze, Regulation of cell division in plants: an Arabidopsis perspective. Prog Cell Cycle Res, 1997. 3: p. 29-41

4. Mironov, V., et al., Cyclin-dependent kinases and cell division in plants—the nexus. Plant Cell, 1999. 11(4): p. 509-522

5. Hemerly, A. S., et al., cdc2a expression in Arabidopsis is linked with competence for cell division. Plant Cell, 1993. 5(12): p. 1711-1723

6. Martinez, M. C., et al., Spatial pattern of cdc2 expression in relation to meristem activity and cell proliferation during plant development. Proc Natl Acad Sci USA, 1992. 89(16): p. 7360-7364

7. Soni, R., et al., A family of cyclin D homologs from plants differentially controlled by growth regulators and containing the conserved retinoblastoma protein interaction motif. Plant Cell, 1995. 7(1): p. 85-103

8. Doerner, P., et al., Control of root growth and development by cyclin expression. Nature, 1996. 380(6574): p. 520-523

9. Colon-Carmona, A., et al., Technical advance: spatio-temporal analysis of mitotic activity with a labile cyclin-GUS fusion protein. Plant J, 1999. 20(4): p. 503-508

10. Fire, A., et al., Potent and specific genetic interference by double-stranded RNA in Caenorhabditis elegans. Nature, 1998. 391(6669): p. 806-811

11. Wesley, S. V., et al., Construct design for efficient, effective and high-throughput gene silencing in plants. Plant J, 2001. 27(6): p. 581-59

12. de Jager, S. M. and J. A. Murray, Retinoblastoma proteins in plants. Plant Mol Biol, 1999. 41(3): p. 295-299

13. Xie, Q., et al., Plant cells contain a novel member of the retinoblastoma family of growth regulatory proteins. Embo 3, 1996. 15(18): p. 4900-4908

14 Vos, P., et al., AFLP: a new technique for DNA fingerprinting. Nucleic Acids Res, 1995. 23(21): p. 4407-4414

15. Breyne, P., et al., AFLP analysis of genetic diversity within and between Arabidopsis thaliana ecotypes. Mol Gen Genet, 1999. 261(4-5): p. 627-634

16. www.arabidopsis.org, arabidopsis home page

17. http://www.pgreen.ac.uk/, pGreen web site

18. Azpiroz-Leehan, R. and K. A. Feldmann, T-DNA insertion mutagenesis in Arabidopsis: going back and forth. Trends Genet, 1997. 13(4): p. 152-156

19. http://www.mcdb.ucla.edu/Research/Jacobsen/EMSmutagenesis.html, EMS mutagenesis of Arabidopsis

20. www.arabidopsis.org, TAIR the Arabidopsis database

1. A method of identifying in a eukaryotic target organism a nucleic acid sequence encoding a product that is involved in cell growth regulation in said target organism, the method comprising the steps of:
(a) providing a plant that is genetically modified to have tissue exhibiting, relative to the tissue of its non-genetically modified parent plant, accelerated growth,
(b) subjecting a multiplicity of the genetically modified plant, or parts thereof to a mutagenisation treatment,
(c) selecting from the thus treated plants or parts thereof mutant plants having, relative to the wild type plant or the genetically modified plant of step (a), a phenotype characterised by an altered morphological structure or an altered colour,
(d) identifying in said selected mutant plants nucleic acid sequence(s) having a nucleic acid sequence which is different from the corresponding sequence(s) in a non-mutagenised wild type plant or the genetically modified plant, and, using said nucleic acid sequence(s),
(e) identifying in the eukaryotic target organism a target nucleic acid sequence comprising a sequence encoding a product that is involved in cell growth regulation.

2. A method according to claim 1, wherein the wild type plant or the parent plant for the genetically modified plant of step (a) is selected from a group consisting of Lotus japonicus, Medicago truncatula, Oryza sativa, Antirrhinum majus and Arabidopsis thaliana.

3. A method according to claim 2, wherein the wild type plant or the parent plant is Arabidopsis thaliana.

4. A method according to claim 1, wherein the accelerated growth of tissue of the genetically modified plant is due to overexpression of a gene selected from the group consisting of a gene coding for a cyclin, a gene coding for a transcription factor including E1A, E2F, myc, and any other gene positively affecting the cell cycle regulatory system.

5. A method according to claim 4, wherein the gene coding for a cyclin is selected from the group consisting of a gene encoding a cyclin of B-type and a gene encoding a cyclin of D type.

6. A method according to claim 1, wherein the genetically modified plant is obtained by introducing into a cell of the parent plant a gene construct comprising a promoter and, operably linked thereto, a nucleotide sequence encoding a gene product that is involved in acceleration of growth in a tissue of the thus modified plant.

7. A method according to claim 6, wherein said gene product activates the cell cycle regulatory system of the plant.

8. A method according to claim 7, wherein the gene product is encoded by a gene selected from the group consisting of a gene coding for a cyclin, a gene coding for a transcription factor including E2F and myc, and any other gene positively affecting the cell cycle regulatory system.

9. A method according to claim 8, wherein the gene coding for cyclin is selected from the group consisting of the cyc1At gene (encoding a mitotic cyclin of B-type), the AtcycD2 gene (encoding a G1 cyclin of D-type) and the AtcycD1 gene (also encoding a G1 cyclin).

10. A method according to claim 6, wherein the promoter is a plant gene promoter.

11. A method according to claim 10, wherein the promoter is selected from the group consisting of an inducible promoter and a constitutive promoter.

12. A method according to claim 10, wherein the plant gene promoter is selected from the group consisting of an Atcdc2a promoter (prAtcdc2a), a 35S promoter and an Atcdc2b-promoter.

13. A method according to claim 6 wherein the gene construct comprises a poly-adenylation site.

14. A method according to claim 13, wherein the poly-adenylation site is derived from the Nopaline synthetase gene of Agrobacterium tumefaciens, an octopine synthetase gene or 35S polyadenylation sequences.

15. A method according to claim 6, wherein the gene construct is introduced by means of Agrobacterium tumefaciens or Agrobacterium rhizogenez.

16. A method according to claim 1, wherein the mutagenisation treatment of step (b) is performed by a method selected from the group consisting of EMS mutagenesis, T-DNA-mutagenesis and mutagenesis by using a transposable element.

17. A method according to claim 1, wherein the identification in step (d) of nucleic acid sequence(s) having a sequence which is different from the corresponding sequence(s) in the non-mutagenised transgenic plant is performed using a method selected from the group consisting of an Amplified Fragment Length Polymorphism (AFLP) method, a Single Sequence Length Polymorphism (SSLP), a differential display method, a restriction fragment length polymorphism (RFLP) method, a Single Strand Conformation Polymorphism (SSCP) method, allele specific amplification, restriction PCR, PCR, sequencing and a Single Nucleotide Polymorphism (SNP) method.

18. A method according to claim 17, wherein the identification method is an SSLP method.

19. A method according to claim 1, wherein the nucleic acid sequence identified in step (d) and/or the product encoded by the sequence is functionally associated with the phenotype of the selected mutant plants of step (c).

20. A method according to claim 1, wherein, in step (e), the target nucleic acid sequence is identified by a homology search in a genome database for the target organism or by molecular probing.

21. A method according to claim 20, wherein the molecular probing is carried out using a method selected from the group consisting of PCR, northern blotting, Southern blotting, arraying and direct sequencing.

22. A method according to claim 1, comprising the further step of isolating the target nucleic acid sequence identified in step (e).

23. A method according to claim 1, wherein the product of the target nucleic acid sequence is functionally active in a signal transduction cascade leading to suppression of cell growth in the target organism.

24. A method according to claim 1, wherein the product of the target nucleic acid sequence is a suppressor of cell growth in the target organism.

25. A method according to claim 1, wherein a putative functional association between the plant nucleic acid sequence identified in step (d) and the target nucleic acid sequence is determined by homology analysis between said plant nucleic sequence and said target nucleic sequence.

26. A method according to claim 1, wherein a putative functional association between the plant nucleic acid sequence identified in step (d) and the target nucleic acid sequence is determined by analysing the effect of expressing the target nucleic acid sequence in an in vitro model for assaying cell growth regulation activity.

27. A method according to claim 1, wherein a putative functional association between the plant nucleic acid sequence identified in step (d) and the target nucleic acid sequence is determined by analysing the effect of expressing the target nucleic acid sequence in an in vivo model for assaying cell growth regulation activity.

28. A method according to claim 1, wherein the eukaryotic target organism is a cell selected from the group consisting of a microbial cell, a plant cell and a mammalian cell.

29. A method according to claim 28, wherein the microbial cell is a yeast cell.

30. A method according to claim 28, wherein the mammalian cell is a cell of a mammal selected from the group consisting of insects, birds, mice, rats, guinea pigs, cats, dogs, apes, primates including humans.

31. A method of determining the tumour suppressor activity, if any, of a gene product encoded by a eukaryotic cell gene, the method comprising the steps of:
(a) providing a wild type plant or a plant that is genetically modified to have tissue exhibiting, relative to the tissue of its non-genetically modified parent plant, accelerated growth,
(b) subjecting a multiplicity of the wild type plant or the genetically modified plant or parts thereof to a mutagenisation treatment,
(c) selecting from the thus treated plants or parts thereof a mutant plant having, relative to the wild type plant or the genetically modified plant of step (a), a phenotype characterised by an altered morphological structure or an altered colour,
(d) identifying in said selected mutant plant a nucleic acid sequence having a sequence which is different from the corresponding sequence in the non-mutagenised wild type plant or genetically modified plant, and, using said different nucleic acid, identifying in the eukaryotic cell a homologue or analogue gene putatively involved in cell cycle regulation,
(e) transforming the coding sequence of said homologue or analogue gene into a mutant plant of step (c) under conditions permitting the sequence to be expressed, and
(f) determining whether or not the thus transformed mutant plant reverts to its wild type phenotype, such reversion being indicative of tumour suppressor activity of the homologue or analogue gene product.


SEQUENCE LISTING (9 sequences)

SEQ ID NO: 1 - DNA, 21 bp, Arabidopsis thaliana
aagttttgtcgacatatatat

SEQ ID NO: 2 - DNA, 21 bp, Arabidopsis thaliana
aacctgatccatggattcctg

SEQ ID NO: 3 - DNA, 645 bp, Arabidopsis thaliana
atggctgagaatcttgcttgtggtgaaaccagcgagtcatggatcattgacaacgacgatgatgatatcaactatggcggcggatttacgaacgagattgattacaatcaccaactttttgctaaagacgacaactttggcggcaacggatcaattccgatgatgggttcttcttcatcgtccttgagtgaagacagaatcaaagagatgttggtgagagagattgagttttgccctggaactgattatgttaagagattgctttctggtgatttggatttgtctgttcgaaaccaagctcttgattggattctaaaggtatgtgatttcttcttttgattgctttgatctgtgttgtgttttaagacaaacctcaaatctggtttggtctctttttgtgctttgtgattgagtttaagtgtgttctttgatcagtttgatagctcattcattctctgatgtatttattatagattaattgaatcttctctatagattgaggtattgatcttgttgagtaattgagttcttttgtctttaatgccaattgttctacaggtttgtgctcattaccattttggacatctgtgcatatgcctatccatgaactacttggatcggttcttaacatcctatgaattgccg

SEQ ID NO: 4 - DNA, 34 bp, Arabidopsis thaliana
cgcggatccgaagagcacacataatattggaagc

SEQ ID NO: 5 - DNA, 33 bp, Arabidopsis thaliana
ccatcgatcagatgtcgtctcaactctctaggc

SEQ ID NO: 6 - DNA, 33 bp, Arabidopsis thaliana
cggggtaccagatgtcgtctcaactctctaggc

SEQ ID NO: 7 - DNA, 36 bp, Arabidopsis thaliana
gccgctcgagcgaagagcacacataatattggaagc

SEQ ID NO: 8 - DNA, 16 bp, Arabidopsis thaliana
gatgagtcctgagtaa

SEQ ID NO: 9 - DNA, 16 bp, Arabidopsis thaliana
gactgcgtaccaattc